(19) United States
(12) Patent Application Publication
(10) Pub. No.: US 2024/0378816 A1
(43) Pub. Date: Nov. 14, 2024

(54) DATA OPTIMIZATION METHODS FOR DYNAMIC CUT BOUNDARY

(71) Applicant: Monogram Orthopaedics Inc., Austin, TX (US)

(72) Inventor: Kamran SHAMAEI, Austin, TX (US)

(73) Assignee: Monogram Orthopaedics Inc., Austin, TX (US)

(21) Appl. No.: 18/763,095

(22) Filed: Jul. 3, 2024

Related U.S. Application Data
(63) Continuation of application No. PCT/US2023/060066, filed on Jan. 4, 2023.
(60) Provisional application No. 63/266,471, filed on Jan. 6, 2022.

(51) Int. Cl. G06T 19/00; G06T 15/30; G16H 20/40; G16H 50/50

(52) U.S. Cl. CPC G06T 19/003; G06T 15/30; G16H 20/40; G16H 50/50; G06T 2210/41

(57) ABSTRACT

A data optimization method for a dynamic cut boundary includes obtaining a three-dimensional (3D) model, of a patient anatomy, that comprises a collection of points defining a periphery thereof; based on clipping the 3D model to form clipped surface(s) intersecting the periphery of the 3D model, obtaining a set of points that are on/adjacent intersection(s) of the clipped surface(s) and the periphery of the 3D model, the set of points forming a path along the periphery of the 3D model and corresponding to points on the patient anatomy; determining an ordering, of the set of points, that specifies a sequence of the points from a first point to a last point in a continuous path along the periphery of the 3D model, such that each next point in the sequence is a next-encountered point traveling along the continuous path; and performing processing based on the determining.

### BACKGROUND

Some surgical workflows require manipulations of rendered three-dimensional (3D) objects, specifically, patient anatomical (e.g. bone) models, reconstructed from a CT scan or other medical imaging. Such manipulations may include, by way of non-limiting example, planning resections to accommodate implant placement for joint reconstruction. For surgical robotics applications, it may be desirable to command a robot to execute these resections or to have a user perform them but to constrain such execution (for example, with a robot imposing virtual boundaries).

### SUMMARY

Shortcomings of the prior art are overcome and additional advantages are provided through the provision of a computer-implemented method. The method includes obtaining a three-dimensional (3D) model of a patient anatomy, the 3D model comprising a collection of points defining a periphery of the 3D model, the periphery of the 3D model representing a periphery of the patient anatomy; based on clipping the 3D model, the clipping forming at least one clipped surface of the 3D model that intersects the periphery of the 3D model, obtaining a set of points, of the collection of points, that are on or adjacent one or more intersections of the at least one clipped surface and the periphery of the 3D model, the set of points forming a path along the periphery of the 3D model and corresponding to points on the patient anatomy; determining an ordering of the set of points, the ordering specifying a sequence of the points, of the set of points, from a first point of the set of points to a last point of the set of points in a continuous path along the periphery of the 3D model, such that each next point in the sequence from a current point in the sequence is a next-encountered point traveling along the continuous path from the first point to the last point; and performing processing based on the determining.

Further, a computer system is provided that includes a memory and a processor in communication with the memory, wherein the computer system is configured to perform a method. The method includes obtaining a three-dimensional (3D) model of a patient anatomy, the 3D model comprising a collection of points defining a periphery of the 3D model, the periphery of the 3D model representing a periphery of the patient anatomy; based on clipping the 3D model, the clipping forming at least one clipped surface of the 3D model that intersects the periphery of the 3D model, obtaining a set of points, of the collection of points, that are on or adjacent one or more intersections of the at least one clipped surface and the periphery of the 3D model, the set of points forming a path along the periphery of the 3D model and corresponding to points on the patient anatomy; determining an ordering of the set of points, the ordering specifying a sequence of the points, of the set of points, from a first point of the set of points to a last point of the set of points in a continuous path along the periphery of the 3D model, such that each next point in the sequence from a current point in the sequence is a next-encountered point traveling along the continuous path from the first point to the last point; and performing processing based on the determining.

Yet further, a computer program product including a computer readable storage medium readable by a processing circuit and storing instructions for execution by the processing circuit is provided for performing a method. The method includes obtaining a three-dimensional (3D) model of a patient anatomy, the 3D model comprising a collection of points defining a periphery of the 3D model, the periphery of the 3D model representing a periphery of the patient anatomy; based on clipping the 3D model, the clipping forming at least one clipped surface of the 3D model that intersects the periphery of the 3D model, obtaining a set of points, of the collection of points, that are on or adjacent one or more intersections of the at least one clipped surface and the periphery of the 3D model, the set of points forming a path along the periphery of the 3D model and corresponding to points on the patient anatomy; determining an ordering of the set of points, the ordering specifying a sequence of the points, of the set of points, from a first point of the set of points to a last point of the set of points in a continuous path along the periphery of the 3D model, such that each next point in the sequence from a current point in the sequence is a next-encountered point traveling along the continuous path from the first point to the last point; and performing processing based on the determining.

In embodiments, the obtained set of points comprises the points, of the set of points, with no ordering specified in terms of the sequence from the first point to the last point in the continuous path. In embodiments, the obtained set of points is obtained without a specified ordering of the points. In embodiments, the obtained set of points is obtained with an order that indicates two points of the set, having one or more other points of the set between the two points along the periphery of the 3D model by being sequentially-prior, in the order, to the one or more other points. In embodiments, the ordering of the set of points identifies a contiguous path on the periphery of the patient anatomy identifying step-wise traversal around the patient anatomy in a direction.

In embodiments, the method further includes receiving from a user a definition of one or more clips to clip the 3D model. In embodiments, the method further includes obtaining a definition of one or more clips to clip the 3D model and determining the set of points by clipping the 3D model with the defined one or more clips. In embodiments, the clipping is defined by one or more clips defined by a user.

In embodiments, the set of points comprises at least 5 points. In embodiments, the performing processing comprise outputting the ordering of the set of points as, or to facilitate generation of, a cut path along the periphery of the patient anatomy. In embodiments, the further includes using the ordering of the set of points to define a virtual boundary for surgical cutting execution.

Additional features and advantages are realized through the concepts described herein.

### BRIEF DESCRIPTION OF THE DRAWINGS

Aspects described herein are particularly pointed out and may be distinctly claimed, and objects, features, and advantages of the disclosure are apparent from the detailed description herein taken in conjunction with the accompanying drawings in which:

FIG. 1 depicts an example showing flat surfaces of patient bone represented as cross-sections of a three-dimensional (3D) model;

FIG. 2A depicts virtual boundaries and corresponding implants used in conventional approaches that pre-define cut plans;

FIG. 2B depicts a planning screen for virtual implant placement;

FIG. 2C depicts an example of necessary clips to remove bone to properly fit an implant;

FIG. 3 depicts an example illustrating critical soft tissue that may be encountered intraoperatively that the cutting tool must avoid during cutting;

FIG. 4 illustrates deficiencies of generic implants achieving perfect coverage of resected anatomy;

FIG. 5 depicts an example of CT scan output generation;

FIG. 6 depicts an example of a 3D rendered model and a polygon mesh for 3D representation thereof;

FIG. 7 depicts an example continuous path around an object;

FIGS. 8A-8B conceptually depict examples of point misordering when clipping a model;

FIG. 9 depicts an example computer system to incorporate and use aspects

described herein; and

FIGS. 10-16 depict examples to illustrate aspects described herein.

### DETAILED DESCRIPTION

Commanding a robot (or other actor) to execute or assist with surgical cuts may require, by way of non-limiting example, generating a cut path (or “toolpath”) for a tool (e.g. cutting instrument) to follow. A toolpath represents a series of ordered coordinates through which the tool (for example, a cutting tool like a sagittal saw) may travel to perform a function, like cutting. Generally, the tool executes its intended task at or around each ordered coordinate on the toolpath. The coordinates are ordered so that the cutting tool can, starting from a starting point, cut to the sequentially-next point in the series, then the sequentially-next point in the series, and so on, to reach an ending point (which may be the same as the starting point or a different point) and thereby form a continuous path along a surface of the anatomy.

Described herein are approaches for cut path boundary generation, for instance, by way of sorting points on the boundary of a 3D model so that their sorted order provides data that is useful to a cut path generating algorithm.

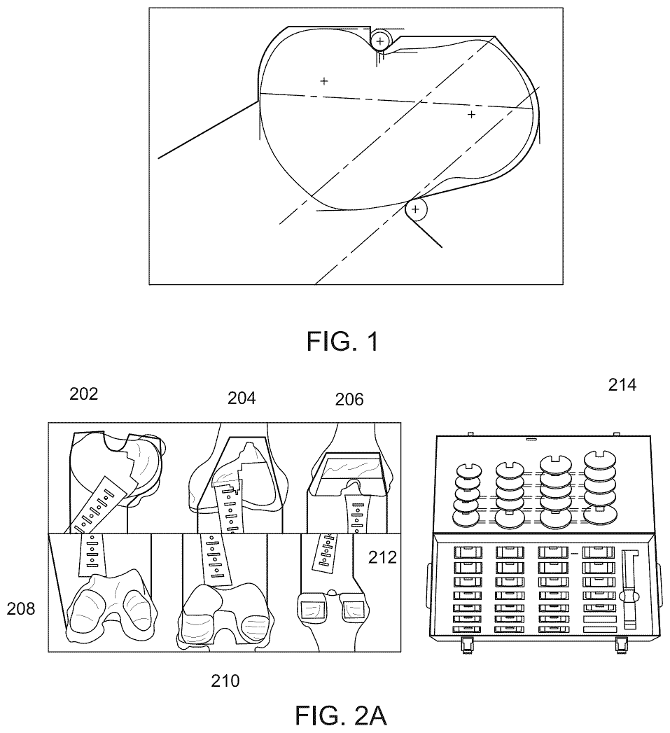

To generate a toolpath, one approach is to clip the 3D model to simulate the desired outcome after cutting and then solve for the cut paths required to obtain this outcome. By way of non-limiting example, if the cutting tool is a sagittal saw with a planar saw blade that extends distally from a drive mechanism that moves in an oscillatory, back-and-forth pattern in a plane in which the blade is aligned, the software could represent the cuts as a sequence of planes that intersect the 3D model. The resultant cross-sections of the clipped model can be used to assess how the cutting tool (sagittal saw here) should be controlled/commanded to produce such outcomes. In an example in which bone is cut to accommodate implant placement and the cutting tool is a sagittal saw, the desired outcome may be flat surfaces represented as cross-sections of the 3D model, as shown in FIG. 1. It is noted that a series of clips may be desired to remove anatomy (bone), for instance a desired resection could be executed with one or more separate planar clips. It is further noted that clips need not be planer; they could be curved, etc. Therefore, cut paths need not be straight lines per se; they could be curved line(s), formed by two straight or curved lines that intersect/touch to form an abrupt change in direction at a vertex, etc.

There is often a clinical need to generate cut paths dynamically. By way of non-limiting example, CT scan imaging does not generally capture soft-tissue structures like ligaments, making pre-operative cut planning difficult, for example to avoid the soft tissue area. In surgery, soft-tissue manipulations may be needed, and the position of resultant cuts may differ than those originally anticipated. Alternately, by way of non-limiting example, a surgeon may find that anatomy (such as a knee) is improperly tensioned during trialing after the planned cuts have been executed, requiring new cuts that have not been pre-planned. Moreover, the pre-planned cut area has no knowledge of the bone geometry, which can be obtained only after an implant is virtually placed on the bone. It may be desired in these situations to seamlessly generate cut paths intraoperatively in order to safely plan and execute unplanned cuts. Aspects described herein can apply to order surface points of the model/anatomy to facilitate dynamic, real-time, accurate cut path generation. Dynamic planning to address this situation can include defining a clip, reordering surface points, creating a cut boundary, and generating the new cut path, for instance.

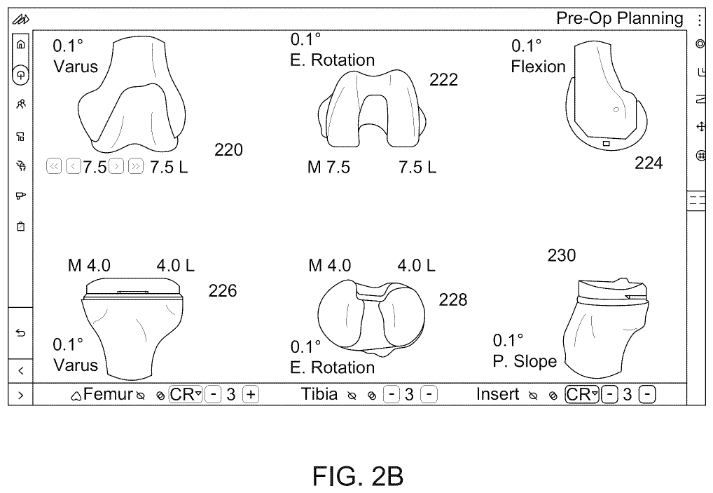

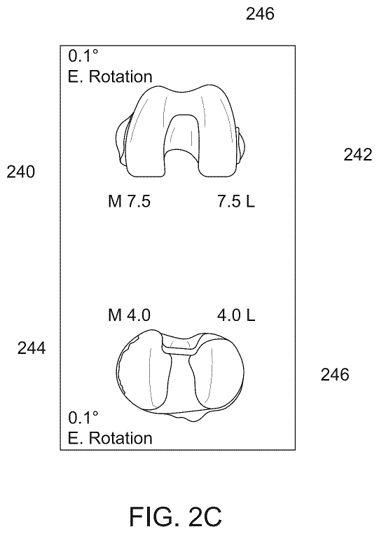

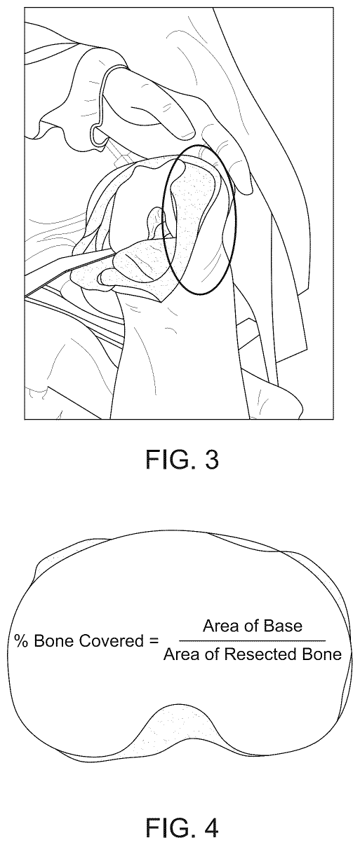

Current state of the art does not dynamically generate tool cut paths in real-time. Cut paths, or virtual boundaries (for instance to prevent the cutting tool from damaging anatomy while cutting) in common commercial offerings are generated offline and correlated to a particular make/model/SKU of an implant or other predefined object to be used. FIG. 2A depicts example virtual boundaries (202, 204, 206, 208, 210, 212) that correspond to selected implants (214). The boundaries are not patient-specific or dynamic but programmed offline. FIG. 2B depicts a planning screen for virtual implant placement. The surgeon is able to manipulate the position of the implants (220, 222, 224, 226, 228, 230) on the one model as shown. FIG. 2C depicts an example of necessary clips (240, 242, 244) to remove bone to properly fit an implant 246. Specifically, a generic implant does not fit perfectly and there are some areas (240, 242, 244) adjacent to the implant 246 that may also need to be clipped.

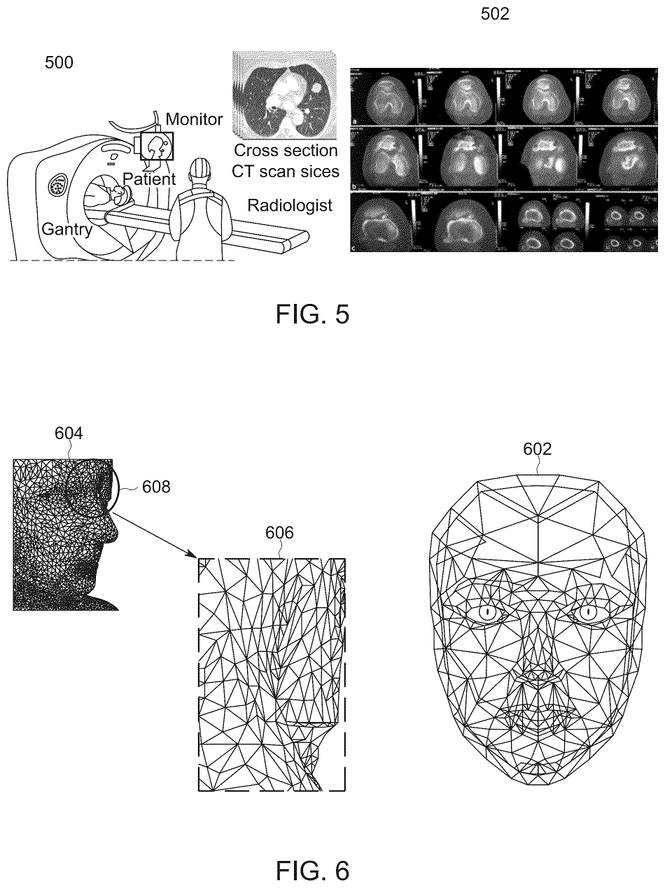

FIG. 3 depicts an example illustrating critical soft tissue (a patellar tendon in this example) that may be encountered intraoperatively that the cutting tool must avoid during cutting. Boundaries protect patient anatomy by managing the proximity of the cutting tool to such structures. A boundary in practice represents the furthest bone extremity beyond which it is generally not desired for the blade to cut. In the noted practice, the boundary is determined by the surgeon virtually placing an implant on the bone model-the implant can in theory have any shape. Because most implants are generic with imperfect coverage, it is generally best practice (with exception(s) such as with the medial side of a partial knee replacement) to remove bone that would otherwise “sit above” the implant-to-bone interface, for example, to prevent notching (for example notching of the anterior femoral cortex during total knee arthroplasty has been implicated as a cause of subsequent periprosthetic supracondylar femoral fracture). Consequently, in clinical practice the boundary is almost always the outer bone surface.

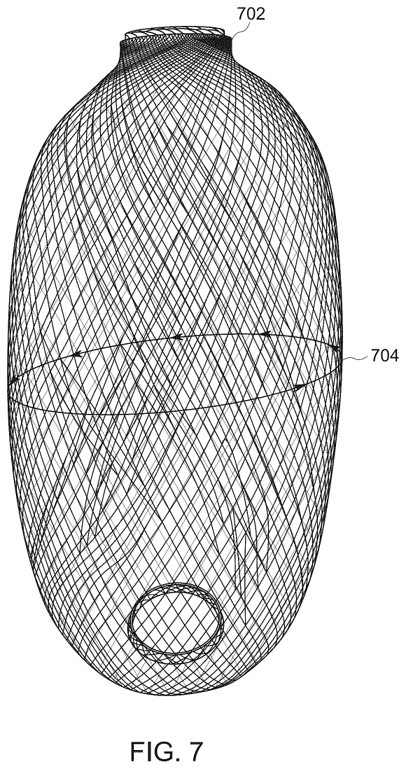

A limitation to the current state of the art is that implants are poor approximations of actual patient anatomy. The implant periphery, for example, does not represent the actual patient-specific bone boundary. Referring to FIG. 4, generic implants do not achieve perfect coverage of the resected bone, and therefore using them to define cut path boundaries is not optimal.

In practice, a general limitation of the above approach to defining cut path boundaries using implants is the frequent occurrence of uncut bone regions (e.g. as in FIG. 2C) that have to be cut by hand after the robotic (or human) execution of initial, planned cut(s). These “air balls” are frustrating to surgeons. It may be possible to correct for uncut regions by expanding the cut boundaries, but this could put critical anatomy at increased risk of damage. An improved approach to generating patient-specific cut path boundaries that represent actual anatomy is therefore desired.

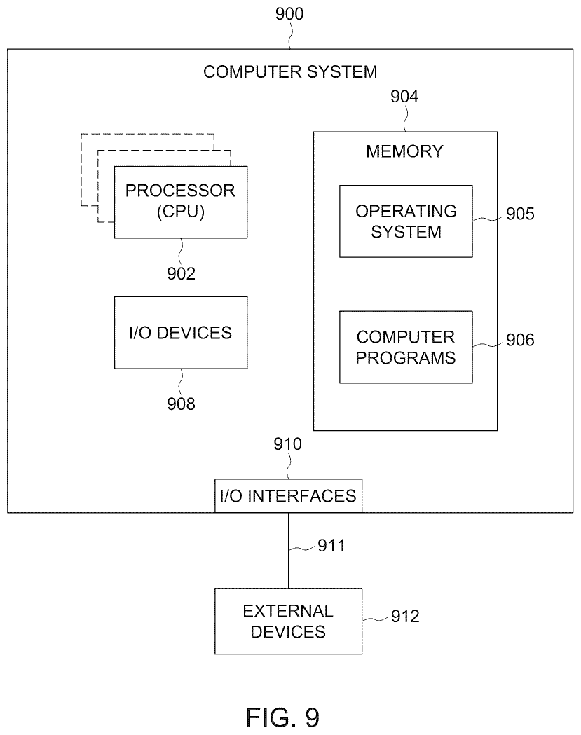

A challenge of creating dynamic cut path boundaries relates to how 3D shapes are rendered by image processing software. To digitally render images with a computer, many 3D computer graphics and image processing software providers use a mesh of flat, multi-sided shapes (such as triangles or other polygons) placed together to represent 3D images. Graphics rendering software often adds colors and textures to these polygons to give the final image a realistic appearance. Generally, the size and number of polygons used to represent a curved object will dictate how smooth and accurate it appears; the smaller and more numerous the polygons, then the more smooth and realistic the object will appear to a viewer. Functionally, many of these rendering tools are designed for optimal 3D rendering rather than, for example, data manipulations such as those for operative planning. FIG. 6 depicts an example of a 3D rendered model of a head. 602 depicts a front view of the head, 604 depicts a front isometric view of the head, and 606 depicts a close-up of a region 608 of the head at the bridge of the nose. Polygons (triangles in this example) used in rendering the head are clearly shown in 606. These are relatively small compared to the polygons used in rendering the head as shown in 602.

More specifically, a problem with off-the-shelf 3D image rendering software is that it is not well suited for data manipulations required for many surgical applications, specifically, and by way of non-limiting example, dynamically defining bone boundaries for cut path generating algorithms. 3D rendering software does not carry useful information when converting 3D image data into a two-dimensional (2D) space. Such conversion may be desired for defining bone boundaries for cut path generation algorithms in which a 3D rendering of a bone (or other anatomy) to be resected is to be processed to provide an accurate 2D cross-section of the bone in a desired plane. This cross-section, and particularly the periphery thereof, is important for clinical and other reasons, for instance to provide a boundary for cut path generation determination. It is not intuitive that 3D rendering software provide a 2D rendering of the 3D object based on an arbitrarily positioned plane through the 3D model.

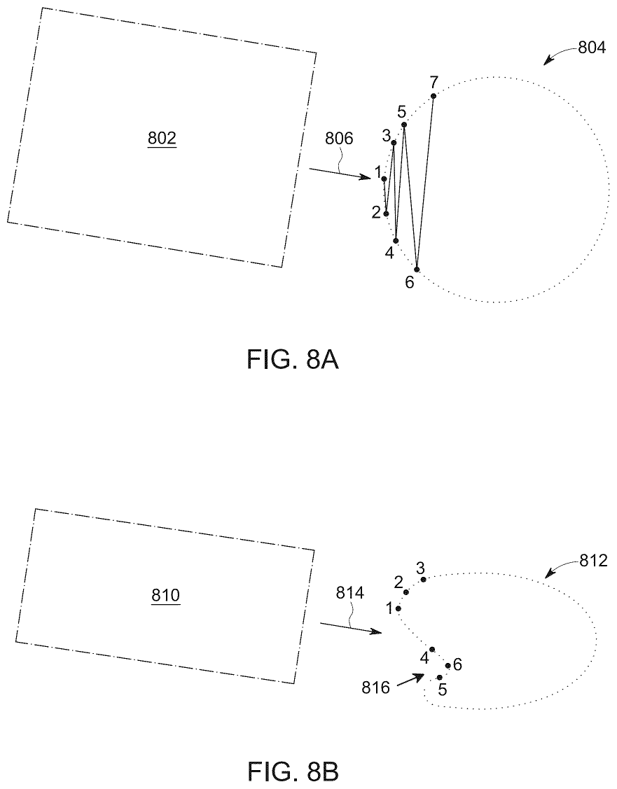

A computed tomography (CT) scan (or other advanced imaging output) is one of the pre-operative inputs to an example surgical workflow to incorporate aspects described herein. Using CT imaging by way of example, a series of 2D images is constructed into a 3D representation/model, as shown in FIG. 5 in which a CT environment captures scan slices (cross-sections) 502. Example operative planning software renders 3D objects as described above using a polygon mesh.

In accordance with one aspect of cut path boundary determination, a 3D model is conceptually clipped with one or more clips (e.g. plane(s) in examples discussed herein but the clips need not be planer) to render resected surface(s). Clipping with a plane (as an example) is analogous to planar cutting with a sagittal saw. The periphery of such a resected surface is the 3D model boundary or edge, which represents the periphery of the anatomy. The features of this periphery are critical for generating tool paths.

Referring to the front view 602 of FIG. 6, the head is a 2D presentation of the 3D object. Because it is a 2D presentation, depth information is not captured when moving in a direction (say horizontally left or right) away from a current point on the surface of the head. For instance, if moving horizontally across the periphery of the 3D rendered model, the next ordered point, or “next-point” may not be the next closest surface point in proximity to the current point in the direction of interest. 3D rendering software displays 3D objects in a 2D plane (a display), so the next point in a given direction may be a point that is not the next closest point in that direction on the periphery. The image processing software prioritizes point ordering for rendering 3D objects rather than for manipulations of the boundaries of clipped models.

A corollary to this can be explained as follows: From the perspective of a viewer of FIG. 6 (looking into the drawing at a perpendicular angle to the front of the 3D model) assume it is desired to push the edge of a plane progressively through the front of the 3D model, with initial contact starting on the bridge of the nose. Say that the plane is flat and perpendicular to the model and to be pushed perpendicularly into the model. This is an example of clipping the model with the plane.

As the plane is pushed toward the model, the edge of the plane will first touch the nose at some initial point. The next point, of the model, that the plane touches as it travels into the model may be on the right side of the nose, followed by a next point on the left side of the nose. In other words, points, of those that define the model, on both sides of the starting point will be encountered the deeper into the image the plane extends.

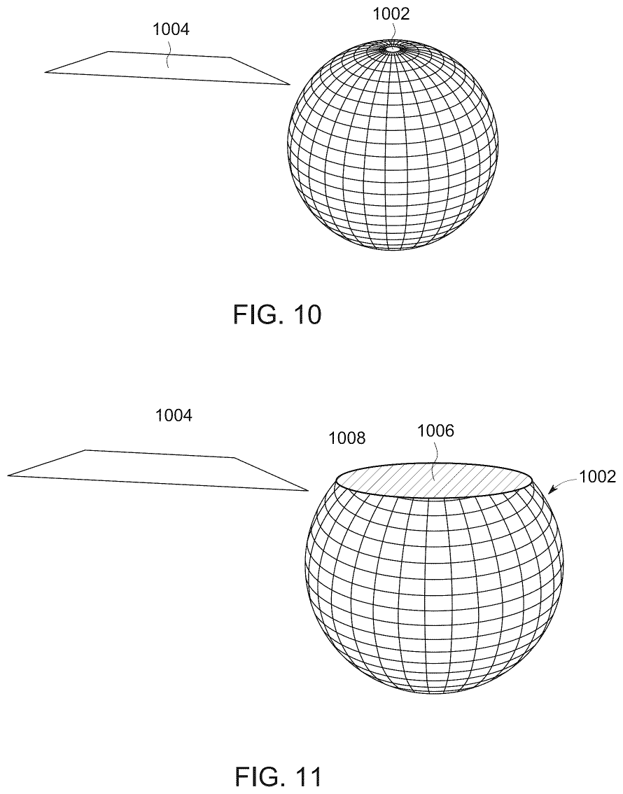

Referring to FIG. 7, shown is a model 702 with a continuous path around the periphery of the object. The continuous path 704 in this example circles all of the way around the object, though a continuous path need not extend all of the way around an object. The path 704 is important in that it identifies the periphery/boundary points of the model that sit on (or adjacent, for instance within some threshold distance) the plane if the plane is intersected with the model to define a clip. The path 704 can also delineate a cut path around the object. A surgeon could cut along this path around the exterior surface of the object, for instance to perform a resection. To figure out exact (or at least closest) points on the model that are on that path, a virtual ‘plane’ may be passed through the model, i.e. to clip the model using image data rendering software. The image processing software might render the planar resection of the 3D object on a 2D display and identify the points around the periphery, but these are unlikely to be ordered in the order of a continuous path (e.g. clockwise or counterclockwise fashion) around the object, meaning ordered in a sequence that traces a continuous path in one direction around the periphery of the model. For example, one cell might render the front portion of the object and the next cell a rear portion.

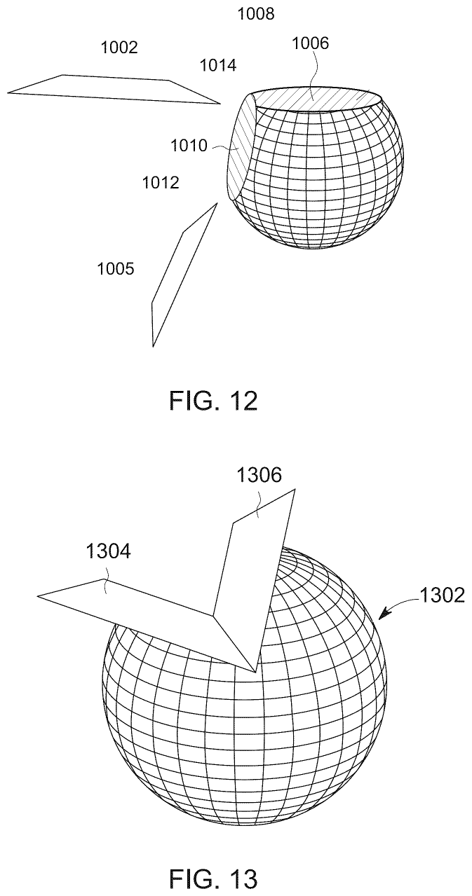

To illustrate, FIGS. 8A-8B conceptually depict examples of point misordering when clipping a model. Referring to FIG. 8A, 802 is a plane (simulating a cutting tool) to clip into a bone 804. A cross-section of bone 804 is shown from an overhead perspective and the bone 804 is a perfect cylinder at the cross-section, in this example. Imagine the plane is to clip in the direction shown by the arrow 806. The clip would be used to identify tens, hundreds, thousands, or millions of points, that define the model, on the periphery of the bone surface, though only seven points, 1-7, on the periphery of the bone surface are labeled in this illustration for ease of understanding. The points are labeled in the order in which the plane encounters them when clipping in the direction shown by arrow 806. Point 1 is the initial point of contact. Point 2 is the next point encountered, followed by point 3, etc. Polygons (termed “cells”) can be formed from these vertices to present a 3D model. For instance, one cell might be a triangle defined by points {4, 5, 6}.

It is desired to order these points in a sequence that, when followed, directs a cutting tool to walk along the surface of the bone connecting point-to-point in a continuous fashion (i.e. forming a continuous path) to produce the desired cut. However, image rendering software is not configured to order these points in such a useful order, for instance one that starts with point 7 and ends with point 6 in the order {7, 5, 3, 1, 2, 4, 6}. In another example, it may be desired to cut all of the way around bone 804, in which case a useful order would be {1, 2, 4, 6, . . . , 7, 5, 3}. This would enable the cutting tool to walk around the periphery in a continuous path from point-to-point, where each point sequentially-next to a prior point is adjacent/next to the prior point.

3D rendering software does not provide such useful information to identify the order of points in such a manner; looking at a 2D rendering (such as 602 of FIG. 6), the next pixel rendered when moving left or right from a point might be farther in the background than a next point. Additionally or alternatively, each next-closest point in a depth direction might be on another side of the object (compare point 4 with point 5 in FIG. 8A for example). If a surgeon were cutting with a list of these points in the order shown, the surgeon would cut a little at point 1, then point 2, then point 3, etc. Eventually the cuts would connect but this is not ideal as the cuts are separate cuts rather than a continuous cut between consecutively, adjacent points.

Referring to FIG. 8B, a similar situation is presented except that the bone 812 is no longer perfectly cylindrical. Again, the plane 810 is to move in the direction indicated by arrow 814. The labeled points 1, . . . , 6 are encountered in the order labeled. It is seen that because of notch 816, point 5 is ordered before point 6 even though a cutting tool moving along the periphery from point 4 to point 5 would encounter point 6 before point 5. In this example, defining the cut to start at point 3 and end at point 5 should properly order the points {3, 2, 1, 4, 6, 5} to form a continuous path.

In both FIGS. 8A and 8B, the labeled points would need to be ordered such that they form a sequential progression that travels (at least partially) around the object, however image rendering software does not provide that functionality. While it might provide a collection of the points that are along the periphery, the order of those would be arbitrary and would not indicate a proper order in terms of walk along/around the object in a sequence of consecutive points to form a continuous path in which each next point to travel to is the next point ahead in the path.

Thus, specific data that may describe characteristics of a model/anatomy periphery, for example, point coordinates, may require reordering to be helpful to a toolpath generating algorithm. Thus, in some aspects, a sorting algorithm is used to order the points on the periphery of a 3D model. The sorted elements may be ordered such that if we sample points in one direction or another (e.g. clockwise or counter-clockwise in the example of FIGS. 8A, 8B) along the periphery, they are ordered in ascending or descending increments. A toolpath represents a series of ordered coordinates through which a tool (for example, a cutting tool like a sagittal saw) may travel to perform a function-it is intuitive that ordering the anatomical model points would simplify the challenge of developing an algorithm that generates toolpaths.

A data manipulation is therefore provided to order a collection of surface points of the 3D model so that their order is informative to a cut path generating algorithm. In a particular example involving polygons for rendering, the sorting algorithm when considering a current point can look at each of one or more candidate next points that are on the periphery of the model and determine a true next point (to travel to from the current point) by considering which next point shares an edge with the current point. Unless the current point is the starting point, it was reached from one vertex with which it shares an edge, and it shares another edge with another vertex of the collection. At the starting point, the direction of travel could be in one direction or another (e.g. around the periphery assuming the cut is to wrap—from a starting point to an ending point that is not the starting point, then there are two candidate starting points—the two ‘end’ vertices, i.e. those that share an edge with just one other point on the periphery line of the model identified from the clip.

By ordering the peripheral points on a patient anatomical model, this enables dynamic generation (virtually and in real-time) of cut path boundaries and virtual boundaries that are related to the actual patient anatomy and not, for example, implants. The workflow enables generation of a truly patient-specific surgical plan intraoperatively. After some cutting, there might a need for a path change, removal of additional anatomy, etc. requiring additional cut(s). Aspects described herein order the points along a periphery defined by a clip in order to develop the appropriate cut path dynamically and in real-time. This is distinct from conventional approaches that define a boundary based on an implant, which is no more than an approximation of where patient anatomy is.

Accordingly, in one aspect an algorithm is provided that dynamically solves for the actual anatomy boundary as an input to a cut path or boundary algorithm, while conventional approaches associate the boundary with implants pre-operatively. Current approaches rely on predefined geometries based on the implant, and can therefore be improved according to aspects described herein to generate patient-specific geometries.

Aspects can be integrated into robotic surgical systems and/or assist in any navigated surgical procedure. Additionally, aspects could have other industrial applications outside of surgical applications.

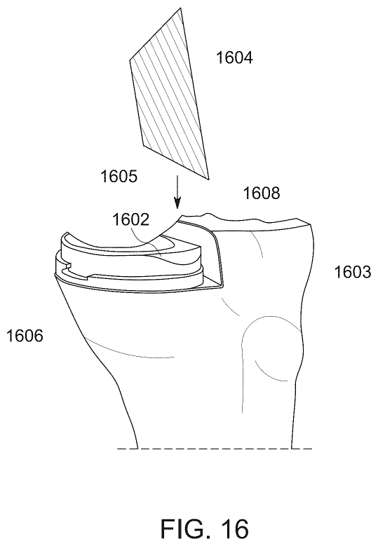

FIGS. 10-16 depict additional examples to illustrate aspects described herein. The examples use a perfect sphere as the 3D model though it is understood that the model of patient anatomy would likely never be perfectly spherical. The model may be of a bone or portion thereof, for instance. The examples also use planar clips for simplicity though it is understood that clips need not be planer; they could be any desired shape.

Referring to FIG. 10, a top portion of the model 1002 is to be clipped. A virtual plane 1004 that defines the clip into the sphere is shown that may be moved toward the model 1002 and intersected with the model 1002 partially or completely through the model. In this example, the resection is to remove completely a top portion of the object 1002. The 3D model 1002 has a surface/periphery to it, which is defined by a collection of points/coordinates. The surface/periphery/boundary of the model 1002 represents the surface/periphery/boundary of the patient anatomy. The plane 1004 represents a blade that will slice around the anatomy (e.g. into the sphere to remove the top).

In FIG. 11, the plane (virtual blade) 1004 sliced around/through the top of the sphere 1002, which creates a new surface, 1006, referred to as a clipped surface. An edge 1008 (forming a circle in the figure) represents the periphery of that clipped surface, which are points on the surface/boundary/periphery of the 3D model. Thus, the edge can be defined by a set of points on the 3D model's surface that form a path along the periphery of the 3D model and correspond to points on the patient anatomy. That set of points is a subset of the collection of points that define the entire surface of the model. 3D rendering software may produce the set of points along that path, but the points will not be in any meaningful order. Aspects described herein order them. Notably, the edge 1008 representing a path in FIG. 11 is what a surgeon/robot could follow to make a proper cut. The portion of clipped surface 1006 contained within the edge 1008 represents bone interior, for instance.

FIG. 12 depicts another example, this time with two clips (both planer). The two clips are made virtually by planes 1004, 1005 to define two clipped surfaces, 1006 and 1010. The edges 1008 and 1012 are the cut paths. 3D rendering software can produce sets of points along those paths but not in a particular, useful order. Aspects described herein can order them. In an example, this is done in two sets of ordered points-one for the cut path 1008 and one for the cut path 1012. In practice, either path 1012 or 1008 may be the first cut and the path will be longer than depicted and circle back to itself. However, the second of the cuts (to form the second surface) would therefore define an edge 1014 shared by both surfaces, which is the intersection of the clips.

FIG. 13 depicts yet another example of two clips 1304, 1306 that are cut into the model 1302 but neither of which pass completely through it. FIG. 14 depicts the corresponding clipped surface result, with two clipped surfaces (surface 1310 and surface 1312) formed by the clipping. The edges 1314, 1316 on the periphery/surface/boundary of the sphere 1302 form the cut paths. The intersection 1318 of the two clips is produced from the surgeon/robot cutting into the sphere.

Thus, based on clipping the 3D model using software, the clipping forms clipped surface(s) of the 3D model that intersect the surface/periphery/boundary of the 3D model. Each clipped surface intersects (e.g. ‘comprises at least one edge terminating at’) the surface of the 3D model. In this and other figures, all of the clipped surfaces extend to the periphery/surface/boundary of the 3D model itself. In FIG. 15 discussed below, for instance, these intersections are the edges 1520, 1522, 1524, 1526.

A process can obtain a set of points, of the collection of points that define the surface of the model, that are on or adjacent (within a threshold distance of) the intersection(s) of the clipped surface(s) and the surface/periphery/boundary of the 3D model. For instance, using FIG. 14 as an example, the software can take the two software-defined clip(s) and identify the points that form the edges. But the 3D model is not made of infinite points and so the software can find the points that are closest (e.g. ‘on or adjacent (within a threshold distance)’) to the true intersection of the model and clipped surfaces depicted as the edges. This produces the set of points that form the edges, though not in a convenient order. The set of points, it is seen when they are connected, form a path along the surface/periphery/boundary of the 3D model and these correspond to points on the patient anatomy;

FIG. 15 shows an example involving three clips. Three clipped surfaces are formed-surface 1528, surface 1530, and a third that is not visible in this view. The edges 1520, 1522, 1524, 1526 on the periphery of the 3D model 1502 are the intersections of the clips with the periphery/surface of the model 1502 and are formed by the points that aspects described herein will obtain and order into a cut path. This could be done in sets for each path—there are four such sets here: one for the curved path/edge 1520 at the top of surface 1528, one for each of the curved paths/edges 1524 and 1526 at the front (foreground) edge of surface 1530 and back (background) edge of surface 1530, and one for the curved path/edge 1522 at the top of the third surface that is not visible in this view. Edge 1532 is an interface between the clipped surfaces 1528 and 1530.

FIG. 16 depicts a virtual plane (1604) that simulates a blade coming generally downward to shave bone 1603 so that an implant 1602 fits. The portion/surface 1605 is bone interior that is being exposed as a result of the clip. Aspects described herein can order the points along the edges 1606 and 1608 in FIG. 16 to define the cut paths to form the two surfaces (one below the implant and surface 1605 adjacent the implant).

Shortcomings of the prior art are overcome and additional advantages are provided through the provision of computer-implemented methods, computer systems configured to perform methods, and computer program products that include computer readable storage media storing instructions for execution to perform methods described herein. Additional features and advantages are realized through the concepts described herein.

In one example of a computer-implemented method, the method includes obtaining a three-dimensional (3D) model of a patient anatomy, the 3D model comprising a collection of points defining a surface/periphery/boundary of the 3D model, the surface/periphery/boundary of the 3D model representing a periphery/boundary/surface of the patient anatomy; based on clipping the 3D model, the clipping forming clipped surface(s) of the 3D model that intersect the surface/periphery/boundary of the 3D model, obtaining a set of points, of the collection of points, that are on or adjacent (within a threshold distance of) the intersection(s) of the clipped surface(s) and the surface/periphery/boundary of the 3D model, the set of points forming a path along the surface/periphery/boundary of the 3D model and corresponding to points on the patient anatomy; determining an ordering of the set of points, the ordering specifying a sequence of the points, of the set of points, from a first point (of the set of points) to a last point (of the set of points) in a continuous path along the periphery of the 3D model, such that each next point in the sequence from a current point in the sequence is a next-encountered point traveling along the continuous path from the first point to the last point; and performing processing based on the determining.

In embodiments, the obtained set of points comprises the points, of the set of points, with no ordering specified in terms of the sequence from the first point to the last point in the continuous path. In embodiments, the obtained set of points is obtained without a specified ordering of the points. In embodiments, the obtained set of points is obtained with an order that indicates two points of the set, having one or more other points of the set between the two points along the periphery of the 3D model by, being sequentially-prior, in the order, to the one or more other points. In embodiments, the ordering of the set of points identifies a contiguous path on the periphery of the patient anatomy identifying step-wise traversal around the patient anatomy in a direction.

In embodiments, the method further includes receiving from a user a definition of one or more clips to clip the 3D model. In embodiments, the method further includes obtaining a definition of one or more clips to clip the 3D model and determining the set of points by clipping the 3D model with the defined one or more clips. In embodiments, the clipping is defined by one or more clips defined by a user.

In embodiments, the set of points comprises at least 5 points. In embodiments, the performing processing comprise outputting the ordering of the set of points as (or to facilitate generation of) a toolpath/cut path along the periphery of the patient anatomy. In embodiments, the further includes using the ordering of the set of points to define a virtual boundary for surgical cutting execution.

It is noted that it may not be even possible, let alone practical, for a human to mentally perform aspects described herein. For instance, models are composed of digital representations of points in space, and performing determinations and ordering of points discussed herein may not be practical or possible in the human mind, let alone at speeds required in surgical and other applications. Aspects described herein at least improve the technical fields of surgical planning, including cut path generation and other technologies.

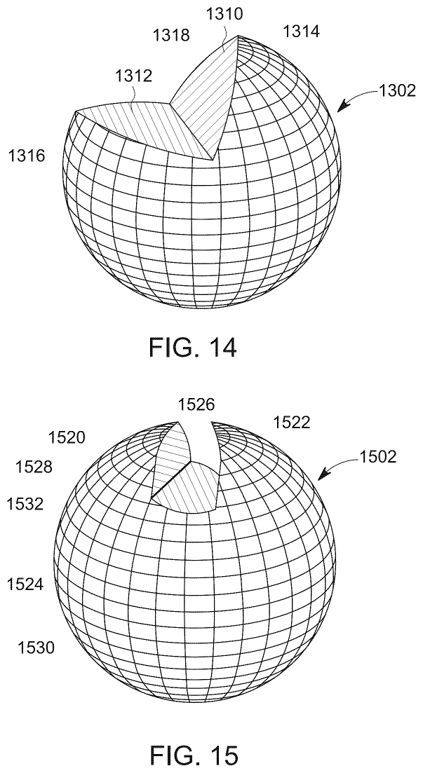

Processes described herein may be performed singly or collectively by one or more computer systems, such as one or more systems that are, or are in communication with, a 3D graphics rendering system/computer, a medical imaging system, a camera system, and/or a tracking system for tracking patient anatomy, as examples. FIG. 9 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) or ARM Holdings plc (Cambridge, England, United Kingdom), as examples.

FIG. 9 shows a computer system 900 in communication with external

device(s) 912. Computer system 900 includes one or more processor(s) 902, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 902 can also include register(s) to be used by one or more of the functional components. Computer system 900 also includes memory 904, input/output (I/O) devices 908, and I/O interfaces 910, which may be coupled to processor(s) 902 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 904 can be or include main or system memory (e.g. Random Access Memory) used in the execution of program instructions, storage device(s) such as hard drive(s), flash media, or optical media as examples, and/or cache memory, as examples. Memory 904 can include, for instance, a cache, such as a shared cache, which may be coupled to local caches (examples include L1 cache, L2 cache, etc.) of processor(s) 902. Additionally, memory 904 may be or include at least one computer program product having a set (e.g., at least one) of program modules, instructions, code or the like that is/are configured to carry out functions of embodiments described herein when executed by one or more processors.

Memory 904 can store an operating system 905 and other computer programs 906, such as one or more computer programs/applications that execute to perform aspects described herein. Specifically, programs/applications can include computer readable program instructions that may be configured to carry out functions of embodiments of aspects described herein.

Examples of I/O devices 908 include but are not limited to microphones, speakers, Global Positioning System (GPS) devices, RGB and/or IR cameras, lights, accelerometers, gyroscopes, magnetometers, sensor devices configured to sense light, proximity, heart rate, body and/or ambient temperature, blood pressure, and/or skin resistance, registration probes and activity monitors. An I/O device may be incorporated into the computer system as shown, though in some embodiments an I/O device may be regarded as an external device (912) coupled to the computer system through one or more I/O interfaces 910.

Computer system 900 may communicate with one or more external devices 912 via one or more I/O interfaces 910. Example external devices include a keyboard, a pointing device, a display, and/or any other devices that enable a user to interact with computer system 900. Other example external devices include any device that enables computer system 900 to communicate with one or more other computing systems or peripheral devices such as a printer. A network interface/adapter is an example I/O interface that enables computer system 900 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 910 and external devices 912 can occur across wired and/or wireless communications link(s) 911, such as Ethernet-based wired or wireless connections. Example wireless connections include cellular, Wi-Fi, Bluetooth®, proximity-based, near-field, or other types of wireless connections. More generally, communications link(s) 911 may be any appropriate wireless and/or wired communication link(s) for communicating data.

Particular external device(s) 912 may include one or more data storage devices, which may store one or more programs, one or more computer readable program instructions, and/or data, etc. Computer system 900 may include and/or be coupled to and in communication with (e.g. as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 900 may be operational with numerous other general purpose or special purpose computing system environments or configurations. Computer system 900 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g. instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method comprising:
obtaining a three-dimensional (3D) model of a patient anatomy, the 3D model comprising a collection of points defining a periphery of the 3D model, the periphery of the 3D model representing a periphery of the patient anatomy;
based on clipping the 3D model, the clipping forming at least one clipped surface of the 3D model that intersects the periphery of the 3D model, obtaining a set of points, of the collection of points, that are on or adjacent one or more intersections of the at least one clipped surface and the periphery of the 3D model, the set of points forming a path along the periphery of the 3D model and corresponding to points on the patient anatomy;
determining an ordering of the set of points, the ordering specifying a sequence of the points, of the set of points, from a first point of the set of points to a last point of the set of points in a continuous path along the periphery of the 3D model, such that each next point in the sequence from a current point in the sequence is a next-encountered point traveling along the continuous path from the first point to the last point; and
performing processing based on the determining.

2. The method of claim 1, wherein the obtained set of points comprises the points, of the set of points, with no ordering specified in terms of the sequence from the first point to the last point in the continuous path.

3. The method of claim 1, wherein the obtained set of points is obtained without a specified ordering of the points.

4. The method of claim 1, wherein the obtained set of points is obtained with an order that indicates two points of the set, having one or more other points of the set between the two points along the periphery of the 3D model by being sequentially-prior, in the order, to the one or more other points.

5. The method of claim 1, wherein the ordering of the set of points identifies a contiguous path on the periphery of the patient anatomy identifying step-wise traversal around the patient anatomy in a direction.

6. The method of claim 1, further comprising receiving from a user a definition of one or more clips to clip the 3D model.

7. The method of claim 1, further comprising obtaining a definition of one or more clips to clip the 3D model and determining the set of points by clipping the 3D model with the defined one or more clips.

8. The method of claim 7, wherein the clipping is defined by one or more clips defined by a user.

9. The method of claim 1, wherein the set of points comprises at least 5 points.

10. The method of claim 1, wherein the performing processing comprises outputting the ordering of the set of points as, or to facilitate generation of, a cut path along the periphery of the patient anatomy.

11. The method of claim 1, further comprising using the ordering of the set of points to define a virtual boundary for surgical cutting execution.

12. A computer system comprising:
a memory; and
a processor in communication with the memory, wherein the computer system is configured to perform a method comprising:
obtaining a three-dimensional (3D) model of a patient anatomy, the 3D model comprising a collection of points defining a periphery of the 3D model, the periphery of the 3D model representing a periphery of the patient anatomy;
based on clipping the 3D model, the clipping forming at least one clipped surface of the 3D model that intersects the periphery of the 3D model, obtaining a set of points, of the collection of points, that are on or adjacent one or more intersections of the at least one clipped surface and the periphery of the 3D model, the set of points forming a path along the periphery of the 3D model and corresponding to points on the patient anatomy;
determining an ordering of the set of points, the ordering specifying a sequence of the points, of the set of points, from a first point of the set of points to a last point of the set of points in a continuous path along the periphery of the 3D model, such that each next point in the sequence from a current point in the sequence is a next-encountered point traveling along the continuous path from the first point to the last point; and
performing processing based on the determining.

13. The computer system of claim 12, wherein the obtained set of points comprises the points, of the set of points, with no ordering specified in terms of the sequence from the first point to the last point in the continuous path.

14. The computer system of claim 12, wherein the obtained set of points is obtained without a specified ordering of the points.

15. The computer system of claim 12, wherein the obtained set of points is obtained with an order that indicates two points of the set, having one or more other points of the set between the two points along the periphery of the 3D model by being sequentially-prior, in the order, to the one or more other points.

16. The computer system of claim 12, wherein the ordering of the set of points identifies a contiguous path on the periphery of the patient anatomy identifying step-wise traversal around the patient anatomy in a direction.

17. The computer system of claim 12, wherein the method further comprises receiving from a user a definition of one or more clips to clip the 3D model.

18. The computer system of claim 12, wherein the method further comprises obtaining a definition of one or more clips to clip the 3D model and determining the set of points by clipping the 3D model with the defined one or more clips.

19. The computer system of claim 18, wherein the clipping is defined by one or more clips defined by a user.

20. The computer system of claim 12, wherein the set of points comprises at least 5 points.

21. The computer system of claim 12, wherein the performing processing comprises outputting the ordering of the set of points as, or to facilitate generation of, a cut path along the periphery of the patient anatomy.

22. The computer system of claim 12, wherein the method further comprises using the ordering of the set of points to define a virtual boundary for surgical cutting execution.

23. A computer program product comprising:
a computer readable storage medium readable by a processing circuit and storing instructions for execution by the processing circuit for performing a method comprising:
obtaining a three-dimensional (3D) model of a patient anatomy, the 3D model comprising a collection of points defining a periphery of the 3D model, the periphery of the 3D model representing a periphery of the patient anatomy;
based on clipping the 3D model, the clipping forming at least one clipped surface of the 3D model that intersects the periphery of the 3D model, obtaining a set of points, of the collection of points, that are on or adjacent one or more intersections of the at least one clipped surface and the periphery of the 3D model, the set of points forming a path along the periphery of the 3D model and corresponding to points on the patient anatomy;
determining an ordering of the set of points, the ordering specifying a sequence of the points, of the set of points, from a first point of the set of points to a last point of the set of points in a continuous path along the periphery of the 3D model, such that each next point in the sequence from a current point in the sequence is a next-encountered point traveling along the continuous path from the first point to the last point; and
performing processing based on the determining.

24. The computer program product of claim 23, wherein the obtained set of points comprises the points, of the set of points, with no ordering specified in terms of the sequence from the first point to the last point in the continuous path.

25. The computer program product of claim 23, wherein the obtained set of points is obtained without a specified ordering of the points.

26. The computer program product of claim 23, wherein the obtained set of points is obtained with an order that indicates two points of the set, having one or more other points of the set between the two points along the periphery of the 3D model by being sequentially-prior, in the order, to the one or more other points.

27. The computer program product of claim 23, wherein the ordering of the set of points identifies a contiguous path on the periphery of the patient anatomy identifying step-wise traversal around the patient anatomy in a direction.

28. The computer program product of claim 23, wherein the method further comprises receiving from a user a definition of one or more clips to clip the 3D model.

29. The computer program product of claim 23, wherein the method further comprises obtaining a definition of one or more clips to clip the 3D model and determining the set of points by clipping the 3D model with the defined one or more clips.

30. The computer program product of claim 29, wherein the clipping is defined by one or more clips defined by a user.

31. The computer program product of claim 23, wherein the set of points comprises at least 5 points.

32. The computer program product of claim 23, wherein the performing processing comprises outputting the ordering of the set of points as, or to facilitate generation of, a cut path along the periphery of the patient anatomy.

33. The computer program product of claim 23, wherein the method further comprises using the ordering of the set of points to define a virtual boundary for surgical cutting execution.
